(19) United States
(12) Patent Application Publication
(10) Pub. No.: US 2006/0276525 A1
(43) Pub. Date: Dec. 7, 2006

(54) Processes of preparing highly pure telmisartan form A, suitable for pharmaceutical compositions

(76) Inventors: Itai Adin, Beer-Sheva (IL); Carmen Iustain, Beer-Sheva (IL); Michael Brand, RaAnana (IL); Ada Salman, Ramat-Gan (IL); Alexander Weisman, Kiryat-Ekron (IL)

Correspondence Address: Martin D. Moynihan;PRTSI, Inc., P.O. Box 16446, Arlington, VA, 22215, US

(21) Appl. No.: 11/434,755

(22) Filed: May 17, 2006

Related U.S. Application Data
(60) Provisional application No. 60/682,019, filed on May 18, 2005.
(60) Provisional application No. 60/778,854, filed on Mar. 6, 2006.
(60) Provisional application No. 60/791,908, filed on Apr. 14, 2006.

(51) Int. Cl. A61K 31/4184; C07D 403/02

(52) U.S. Cl. 514/394; 548/305.4

(57) ABSTRACT

Processes are disclosed for preparing Telmisartan form A, which is free-flowing and which does not contain electrostatic charge and is thus industrially process-able. The free-flowing Telmisartan form A is prepared by crystallization from a polar organic solvent e.g., DMSO, DMF, DMA, NMP, or water and is suitable for use in pharmaceutical compositions. A process is also disclosed for preparing highly-pure Telmisartan form A by precipitation from aqueous solutions.

### RELATED APPLICATIONS

This application claims the benefit of U.S. Provisional Patent Application Nos. 60/682,019 filed on May 18, 2005, 60/778,854 filed on Mar. 6, 2006, and 60/791,908 filed on Apr. 14, 2006. The contents of the above applications are all incorporated by reference as if fully set forth herein

### FIELD OF THE INVENTION

The invention relates to the solid state chemistry of the drug Telmisartan.

### BACKGROUND OF THE INVENTION

Telmisartan (Compound I below), 4′-[2-n-propyl-4-methyl-6-(1-methylbenzimidazol-2-yl)benzimidazol-1-ylmethyl]biphenyl-2-carboxylic acid, is a non-peptide ATI-subtype angiotensin II receptor antagonist.

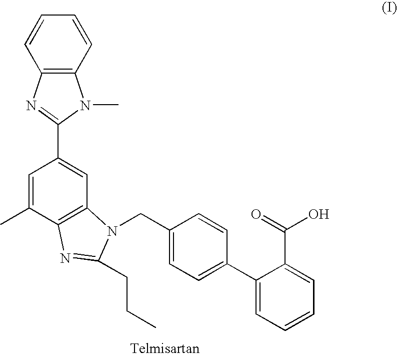

Telmisartan is indicated for treatment of hypertension, either alone or in combination with diuretic agents. It is effective in once-daily dosing, so that significant blood-pressure lowering effect is observed even during the last 6 hours of the dosing interval. Telmisartan is marketed in the US as MICARDIS® and MICARDIS-HCT® by Boehringer Ingelheim.

Telmisartan was first described in U.S. Pat. No. 5,591,762 (hereinafter the '762 patent). According to Example 9 of the '762 patent, Telmisartan was crystallized from acetone and the resulting product had a melting point of 261-263° C.

U.S. Pat. No. 6,358,986 (hereinafter the '986 patent) describes two crystalline forms of Telmisartan denoted as forms A and B. It is stated in the '986 patent that the crystals of Telmisartan polymorph A, which is obtainable according to the prior art, have the shape of long needles. As a result of this crystalline shape, the use of Telmisartan polymorph A in large-scale manufacture, purification, isolation and drying of the material is severely limited.

As alleged in the '986 patent, the process for preparing crystalline Telmisartan form A comprises mixing the material with ethanol, adding activated charcoal and aqueous ammonia and mixing for one hour, then filtering to another stirring apparatus and washing with ethanol. The next step is heating to 70-80° C., adding glacial acetic acid and stirring for further 1.5-2 hours at the same temperature, cooling to 0-10° C., stirring for further 2 hours, isolating the product by centrifugation, washing with ethanol then with water and drying at 70-90° C. According to the detailed description given in the '986 patent, in addition to the disadvantageously prolonged drying process of the Telmisartan form A, very hard particles are obtained. The grinding process of these particles produces a dry powder, which has strong tendency to electrostatic charging and which is virtually impossible to pour and manipulate for pharmaceutical preparations. On the other hand, Telmisartan form B is free from the above mentioned limitations. However, the inventors of the '986 patent could not obtain pure, dry form B because upon drying, some of form B transformed into form A. According to the teachings of the '986 patent, mixtures of Telmisartan form A and form B ranging from 90:10 to 60:40 are suitable for industrial scaling-up, and even a content of 10% of form B is sufficient to ensure that the product will have the positive qualities required for large-scale production.

Therefore, as a consequence of the alleged unsuitability of Telmisartan form A for pharmaceutical use, only a mixture of crystalline Telmisartan form A and form B is claimed in the '986 patent, wherein Telmisartan form A is characterized by having an endothermic maximum at 269±2° C., and Telmisartan form B is characterized by having an endothermic maximum at 183±2° C.

Apparently Telmisartan form A is similar to the original form characterized by its melting point in the '762 patent. The differences between the DSC value and the measured melting point may be attributed to the different methodologies used—the DSC maxima can be slightly different than the visually observed melting point.

Differences in physical properties of crystalline materials (such as flowability) may be caused by different production processes for obtaining these crystalline materials.

Hence, the prior art teaches a lengthy, complicated and industrially disadvantageous process for obtaining crystalline Telmisartan form A.

A mixed solvated-hydrated modification form of Telmisartan, designated as form C, is mentioned in an article by Dinnebier et al., J. Pharm. Sci. 89(11), 2000. Telmisartan form C consists of ⅓ mole equivalent of formic acid and ⅔ mole equivalent of water, which is produced by crystallization from mixtures containing formic acid and water. According to the above mentioned publication, drying of form C leads to pure form B (mentioned above). The article mentions detailed crystallographic data of this form as well as of forms A and B.

The re-crystallization of Telmisartan from N,N-dimethylformamide (DMF) or N,N-dimethylacetamide (DMA) is mentioned in examples 1-3 of the '986 patent. However, since the product is further processed it is believed by the inventors of the present invention that the Telmisartan which is obtained according to the '986 patent, has some defects. Therefore, there is a need in the art for a new process for preparing highly pure Telmisartan form A, which is free-flowing and which does not tend to gain electrostatic charge.

The need to further reprocess the re-crystallized Telmisartan, as taught in the examples of the '986 patent, shows that the product was not highly-pure and/or that it contained residual solvents, because the solvents used therein have high boiling point. By precipitating Telmisartan from an aqueous solution containing acetic acid, as detailed herein, highly pure Telmisartan form A is obtained in high yield e.g., 93%. The obtained Telmisartan form A has low content of residual solvents and is characterized by having a different crystal shape than needles, namely a bulky shape.

### SUMMARY OF THE INVENTION

The present invention provides Telmisartan form A suitable for pharmaceutical use, processes for its preparation and pharmaceutical composition containing the same.

The applicants have surprisingly discovered that upon crystallization of crude Telmisartan from polar organic solvents e.g., dimethyl sulfoxide (DMSO), N,N-dimethylformamide (DMF), N,N-dimethylacetamide (DMA), N-methyl-2-pyrrolidone (NMP), or water an improved crystalline habit appears, having a crystal shape which is entirely different from the shape of long needles. The crystallization process may be carried out without adding a base and without neutralization with an acid.

For example, while crystallizing Telmisartan from dimethyl sulfoxide (DMSO) significant amount of crystals having the shape of prisms was obtained.

The present invention provides a process for producing Telmisartan form A by crystallization from DMSO, the process comprising:

dissolving Telmisartan, obtained as per any suitable method including the method described in reference example 1, in DMSO while heating the mixture to elevated temperature;

cooling the solution for sufficient time to allow crystallization; and

filtering off the crystals, washing and drying.

According to one embodiment, the present invention provides an alternative process for producing Telmisartan form A, having substantial amount of prismatic crystals, by crystallization from water, the process comprising:

dispersing Telmisartan, obtained as per any suitable method including the method described in reference example 1, in hot water, optionally with addition of magnesium stearate;

stirring for an extended time, e.g., for about four days at elevated temperature;

cooling gradually (e.g., slowly) to ambient temperature; and

isolating the crystals by filtration and drying.

According to a preferred embodiment of the present invention, the processes generally described herein cause a considerable change in the crystal shape of the starting material, thus a substantial amount of prismatic crystalline habit is formed (FIG. 1). The product of these processes has better processing properties in comparison to the starting material, such as flowability, and is therefore suitable for pharmaceutical use.

According to another preferred embodiment of the present invention, by crystallizing Telmisartan from DMF, an improved crystalline Telmisartan form A is obtained, having crystal shape which is entirely different from the shape of long needles, described in the '986 patent.

According to another preferred embodiment of the present invention, there are provided improved processes for preparing Telmisartan form A, which is suitable for pharmaceutical use, by crystallization from a polar organic solvent e.g., N,N-dimethylformamide (DMF), N,N-dimethylacetamide (DMA), or N-methyl-2-pyrrolidone (NMP).

According to one aspect of the present invention, the Telmisartan starting material used in the process for preparing Telmisartan form A, may be either in dry state or in wet state.

According to the present invention, the term “dry” means that the material is substantially free of water, while the term “wet” means that the material contains a substantial amount of water.

According to a preferred embodiment of the above-mentioned aspect of the present invention, the process for producing Telmisartan form A by crystallization from a polar organic solvent e.g., N,N-dimethylformamide (DMF), N,N-dimethylacetamide (DMA), or N-methyl-2-pyrrolidone (NMP) using dry Telmisartan, comprises:

providing a mixture of dry Telmisartan, obtained as per any suitable method including the method described in reference example 1, in the polar organic solvent while heating to elevated temperature;

cooling the mixture for sufficient time period to allow crystallization; and

filtering off the crystals, washing and drying, optionally at elevated temperature.

In another preferred embodiment of the above-mentioned aspect of the present invention, there is provided a process for preparing Telmisartan form A by crystallization from a polar organic solvent e.g., N,N-dimethylformamide (DMF), N,N-dimethylacetamide (DMA), or N-methyl-2-pyrrolidone (NMP) using a wet Telmisartan, the process comprising:

dispersing a wet Telmisartan, obtained as per any suitable method including the method described in reference example 1, in a solvent mixture containing toluene and the polar organic solvent;

heating the dispersion to elevated temperature and collecting the water at this temperature by a Dean Stark fitting;

distilling off the toluene and filtering the hot mixture;

cooling the mixture sufficiently and filtering off the crystals; and

washing with an organic solvent and drying the crystals, optionally at elevated temperature.

According to another preferred embodiment of the present invention, upon precipitation of Telmisartan from an aqueous solution containing an acid e.g., acetic acid, highly pure Telmisartan form A is provided in high yield, which is equal to or greater than 93%.

According to another preferred embodiment of the present invention, the Telmisartan form A obtained by precipitation from aqueous solutions containing an acid, e.g., acetic acid has a different crystal shape than needles, namely the form of bulky shape as depicted in FIG. 13, hence the product may be readily filtered, it does not have a tendency to gain electrostatic charge upon grinding, and its flowability makes it suitable for industrial processing and scaling-up.

According to another preferred embodiment of the present invention, the process for producing Telmisartan form A by precipitation from aqueous solutions, comprises:

providing a mixture of Telmisartan, obtained as per any suitable method including the method described in reference example 1, and water while heating;

adding a base and optionally filtering the insoluble matter;

adding an acid to the filtrate to form a suspension and stirring; and

isolating the precipitated crystals.

According to yet another embodiment of the present invention, the Telmisartan form A, prepared essentially as described herein, is solvent free namely it contains residual solvents at a level of less than 5000 ppm, preferably of less than 1000 ppm and has LOD value of less than 0.5%, preferably of less than 0.3%, as measured by means of TGA.

According to yet another embodiment of the present invention, the Telmisartan form A, obtained by the processes described herein, has a purity equal to or greater than 99.5%, and preferably a purity equal to or greater than 99.8%, which makes it suitable for pharmaceutical compositions.

### BRIEF DESCRIPTION OF THE DRAWINGS

In the drawings:

FIG. 1 illustrates an optical microscopy picture of Telmisartan form A prepared by crystallization from water (according to example 2);

FIG. 2 illustrates particle size distribution of Telmisartan form A prepared by crystallization from DMSO (according to example 1);

FIG. 3 illustrates particle size distribution of Telmisartan form A prepared by crystallization from water (according to example 2);

FIG. 4 illustrates XRPD of Telmisartan form A prepared by crystallization from DMSO (according to example 1);

FIG. 5 illustrates XRPD of Telmisartan form A prepared by crystallization from water (according to example 2);

FIG. 6 illustrates TGA of Telmisartan form A prepared by crystallization from DMSO (according to example 1);

FIG. 7 illustrates XRPD of Telmisartan form A prepared by crystallization from DMF (according to example 4);

FIG. 8 illustrates Infra-red spectrum of Telmisartan form A prepared by crystallization from DMF (according to example 4);

FIG. 9 illustrates TGA of Telmisartan form A prepared by crystallization from DMF (according to example 4);

FIG. 10 illustrates DSC of Telmisartan form A prepared by crystallization from DMF (according to example 4);

FIG. 11 illustrates a particle size distribution of Telmisartan form A prepared by crystallization from DMF (according to example 4);

FIG. 12 illustrates an optical microscopy picture of Telmisartan form A prepared by crystallization from DMF (according to example 4); and

FIG. 13 illustrates an optical microscopy picture of Telmisartan form A prepared by precipitation from an aqueous solution (example 7).

### DESCRIPTION OF THE PREFERRED EMBODIMENTS

The present invention provides Telmisartan form A suitable for pharmaceutical use, processes for its preparation and pharmaceutical composition containing the same.

The applicants have surprisingly discovered that upon crystallization of crude Telmisartan from polar organic solvents e.g., dimethyl sulfoxide (DMSO), N,N-dimethylformamide (DMF), N,N-dimethylacetamide (DMA), N-methyl-2-pyrrolidone (NMP), or water an improved crystalline habit appeared, having a crystal shape which is entirely different from the shape of long needles.

For example, while crystallizing Telmisartan from dimethyl sulfoxide (DMSO) significant amount of crystals having the shape of prisms were obtained. This crystal shape is unique because the ratio between the long and narrow dimensions of the crystal is much lower than the characteristic ratio of the needle-shaped Telmisartan crystal mentioned in the literature. Consequently, the physical characteristics of the Telmisartan form A, having prismatic shape crystals, are greatly improved compared to the poor quality of Telmisartan form A mentioned in the literature. The material may be readily filtered, it does not have a tendency to gain electrostatic charge upon grinding, and its flowability makes it suitable for industrial processing and scaling-up.

Thus, according to a preferred embodiment of the present invention, the Telmisartan form A, obtained by crystallization from DMSO, is characterized by having a substantial amount of prismatic crystals with the longest dimension shorter than around 100 μm, as measured by means of optical microscopy.

According to one embodiment of the present invention, the crystalline Telmisartan form A, obtained by crystallization from DMSO, is further characterized by having bulk density of about 0.3 g/ml.

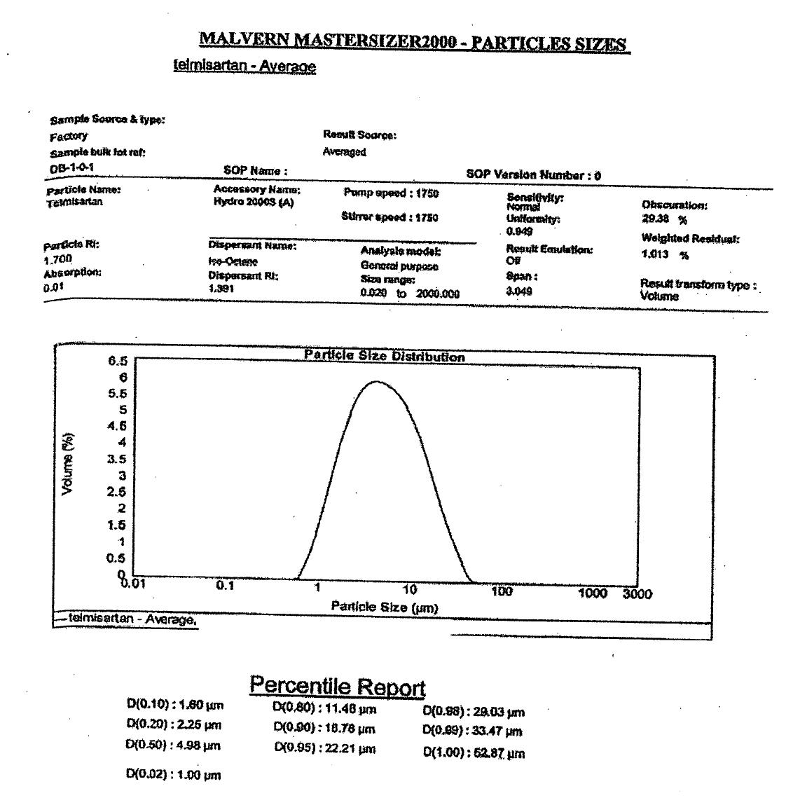

According to another embodiment of the present invention, the crystalline Telmisartan form A, obtained by crystallization from DMSO, is further characterized by an average particle size of about 5μ for 50% of the particles and about 17μ for 90% of the particles, obtained upon milling (FIG. 2).

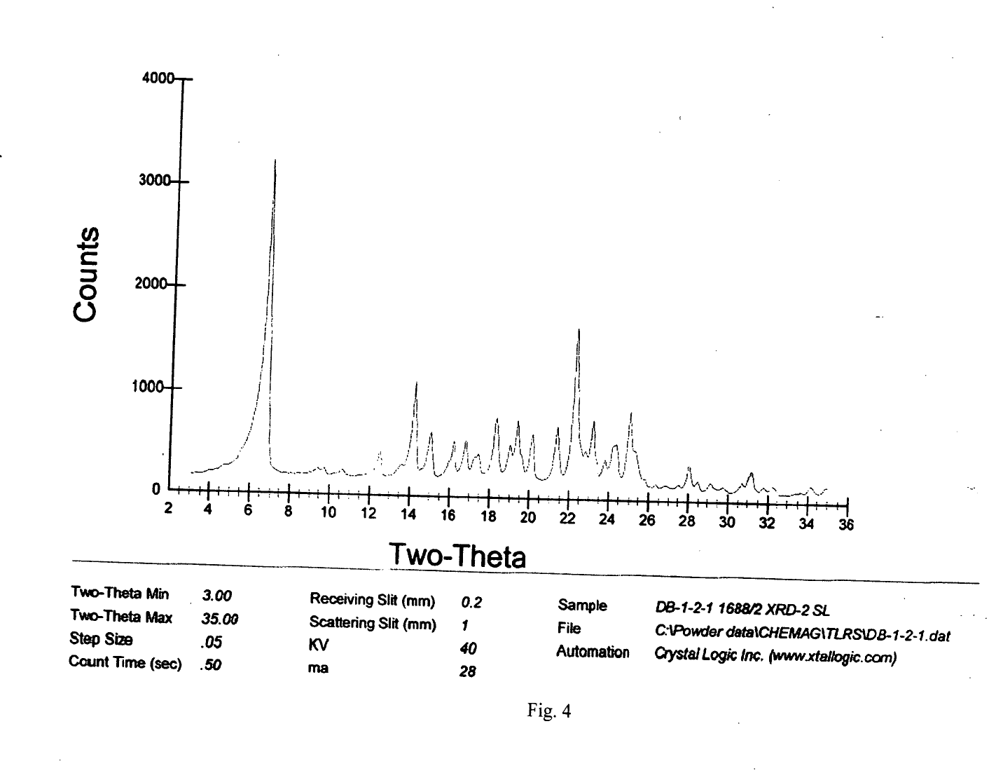

According to another embodiment of the present invention, the crystalline Telmisartan form A, obtained by crystallization from DMSO, is further characterized by having an XRPD spectrum as shown in FIG. 4.

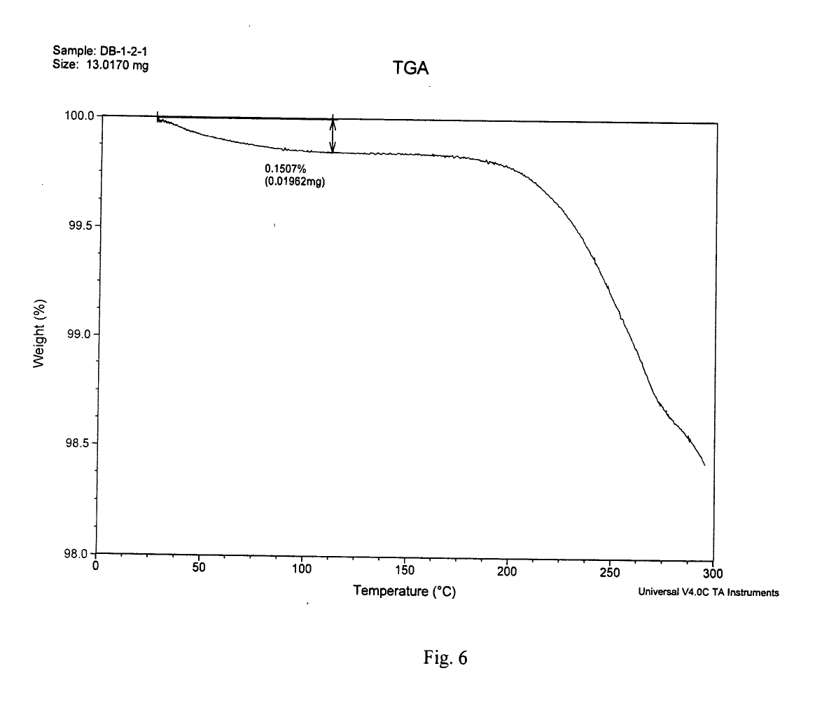

According to another embodiment of the present invention, the crystalline Telmisartan form A, obtained by crystallization from DMSO, is further characterized by having a TGA curve as shown in FIG. 6.

According to another embodiment of the present invention, the purity of Telmisartan form A, obtained by crystallization from DMSO (according to HPLC), is higher than 99.5%, therefore the material complies with pharmaceutical quality and is suitable for pharmaceutical use.

The present invention provides a process for producing Telmisartan form A by crystallization from DMSO, the process comprising:

dissolving Telmisartan, obtained as per any suitable method including the method described in reference example 1, in DMSO while heating the mixture to elevated temperature;

cooling the solution for sufficient time to allow crystallization; and

filtering off the crystals, washing and drying.

According to another embodiment of the present invention, Telmisartan prepared as described herein is not heat-sensitive, therefore the solution of Telmisartan in DMSO is heated to elevated temperature, preferably to about 65° C. and more preferably to about 90° C. or higher. The obtained Telmisartan may be dried at an elevated temperature of at least 50° C., preferably at about 90° C. and more preferably at 100° C. or higher for at least two hours under vacuum.

According to another embodiment of the present invention, Telmisartan form A, prepared essentially as described herein, contains residual DMSO at a level of less than 5000 ppm, preferably of less than 1000 ppm and has LOD value of less than 0.5%, preferably of less than 0.3%, as measured by means of TGA.

The present invention provides an alternative process for producing Telmisartan form A, having substantial amount of prismatic crystals, by crystallization from water, the process comprising:

dispersing Telmisartan, obtained as per any suitable method including the method described in reference example 1, in hot water, optionally with addition of magnesium stearate;

stirring for an extended time, e.g., for about four days at elevated temperature;

cooling gradually (e.g., slowly) to ambient temperature; and

isolating the crystals by filtration and drying.

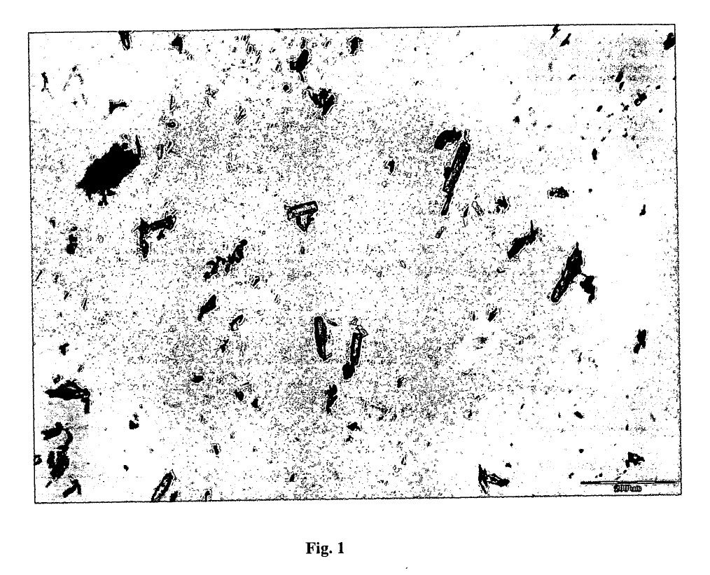

According to a preferred embodiment of the present invention, the process generally described herein causes a considerable change in the crystal shape of the starting material, thus a substantial amount of prismatic crystalline habit is formed (FIG. 1). The product of this process has better processing properties in comparison to the starting material, such as flowability, and is therefore suitable for pharmaceutical use.

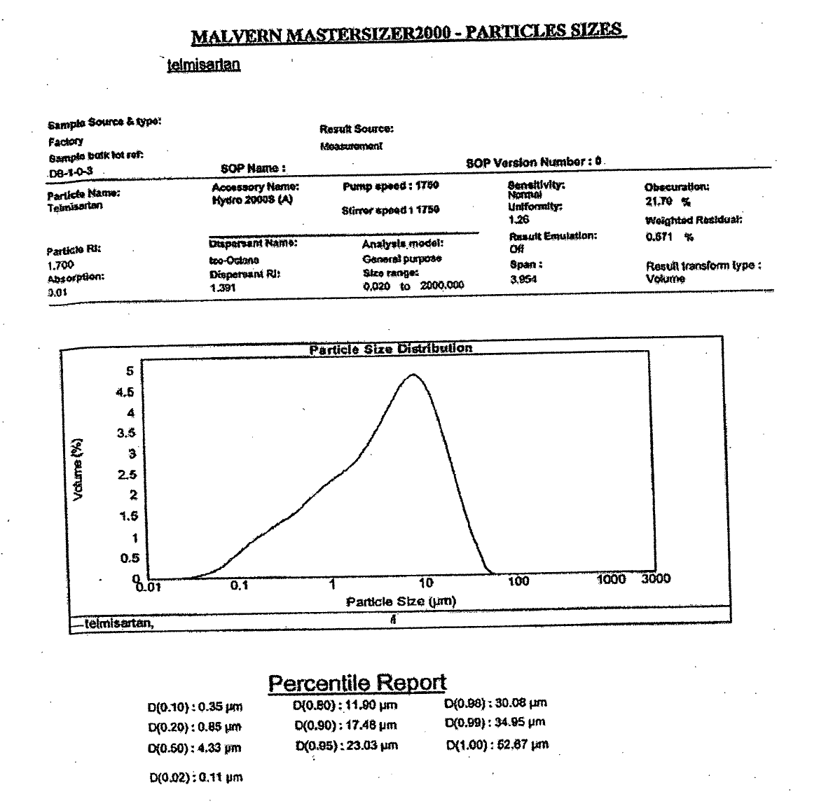

According to another embodiment of the present invention, the crystalline Telmisartan form A, obtained by crystallization from water, is characterized by an average size of about 4.3μ for 50% of the particles and about 17.5μ for 90% of the particles, obtained upon milling (FIG. 3).

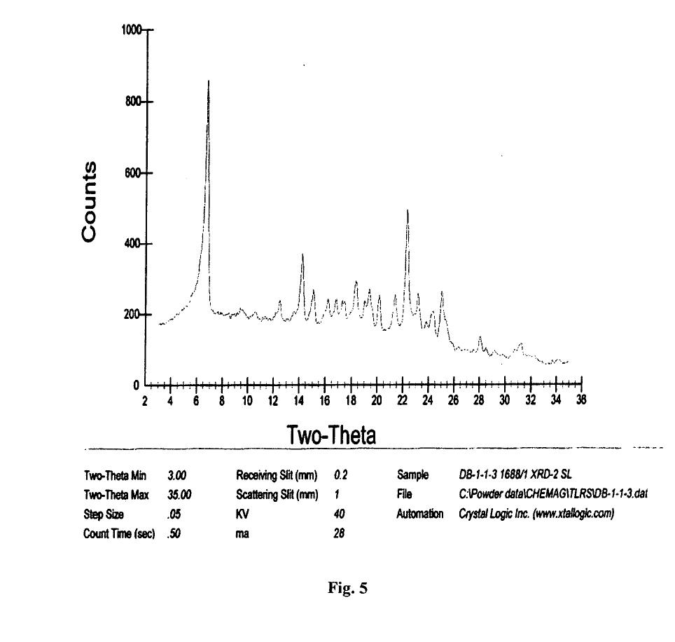

According to another embodiment of the present invention, the crystalline Telmisartan form A, obtained by crystallization from water, is further characterized by having an XRPD spectrum as shown in FIG. 5.

According to another embodiment of the present invention, the crystalline Telmisartan form A, obtained by crystallization from water, is further characterized by having bulk density of about 0.22 g/ml.

According to another embodiment of the present invention, the hot water temperatures range from about 50° C. to reflux, preferably from 80° C. to 90° C. The dispersion of Telmisartan in water is heated to an elevated temperature, preferably about 80-90° C.

According to another embodiment of the present invention, the amount of magnesium stearate used (that may facilitate the dispersion of Telmisartan) is lower than 10%, preferably lower than 5% and more preferably around 1-2%.

According to another embodiment of the present invention, the obtained crystals are dried at elevated temperature, preferably at 50° C., and optionally under vacuum.

According to another preferred embodiment of the present invention, by crystallizing Telmisartan from DMF, an improved crystalline Telmisartan form A is obtained, having crystal shape which is entirely different from the shape of long needles, described in the '986 patent.

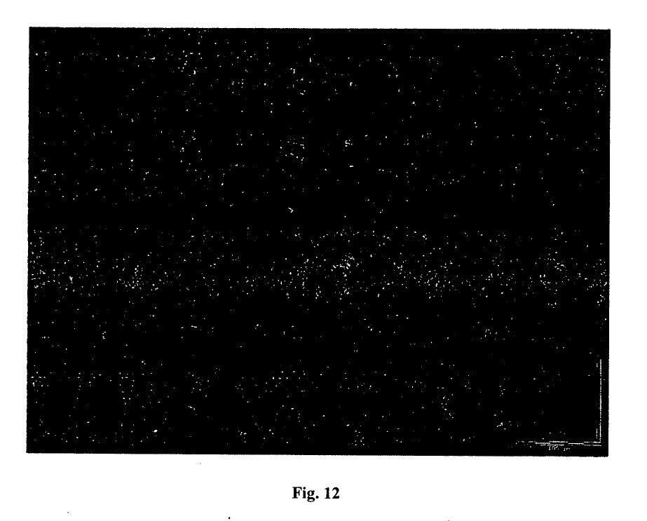

According to one aspect of this embodiment of the present invention, the crystalline Telmisartan form A, obtained by crystallization from DMF, is characterized by an optical microscopy picture, as depicted in FIG. 12, which clearly shows that these particles are not needle-shaped, hence they are free from the limitations, which are described in the '986 patent. Consequently, the crystals of the Telmisartan form A obtained by crystallization from DMF, according to the present invention, are suitable for large-scale manufacture.

According to another embodiment of the present invention, the crystalline Telmisartan form A, obtained by crystallization from DMF, may be readily filtered, it does not have a tendency to gain electrostatic charge upon grinding, and its flowability makes it suitable for industrial processing and scaling-up.

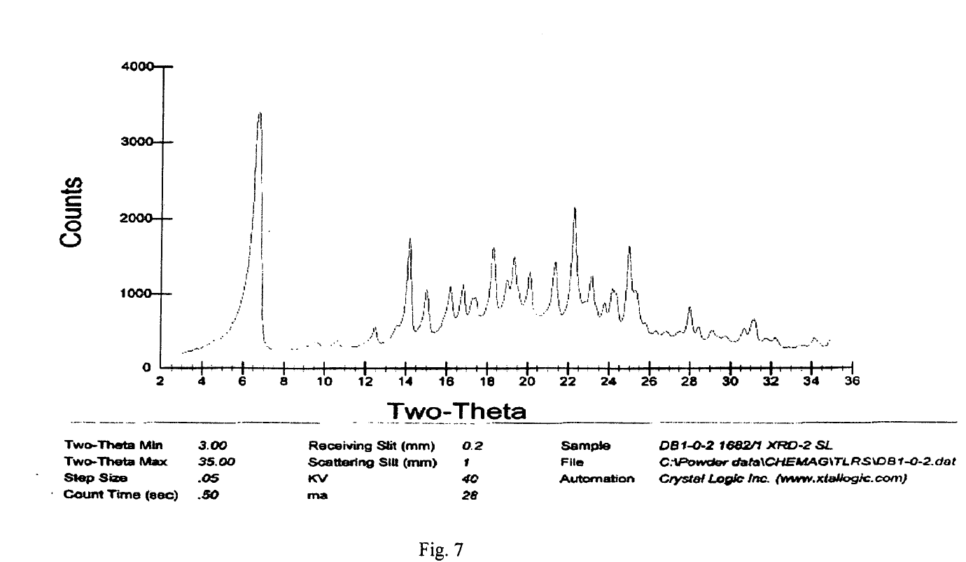

According to another embodiment of the present invention, the crystalline Telmisartan form A, obtained by crystallization from DMF, is further characterized by having a XRPD spectrum as shown in FIG. 7.

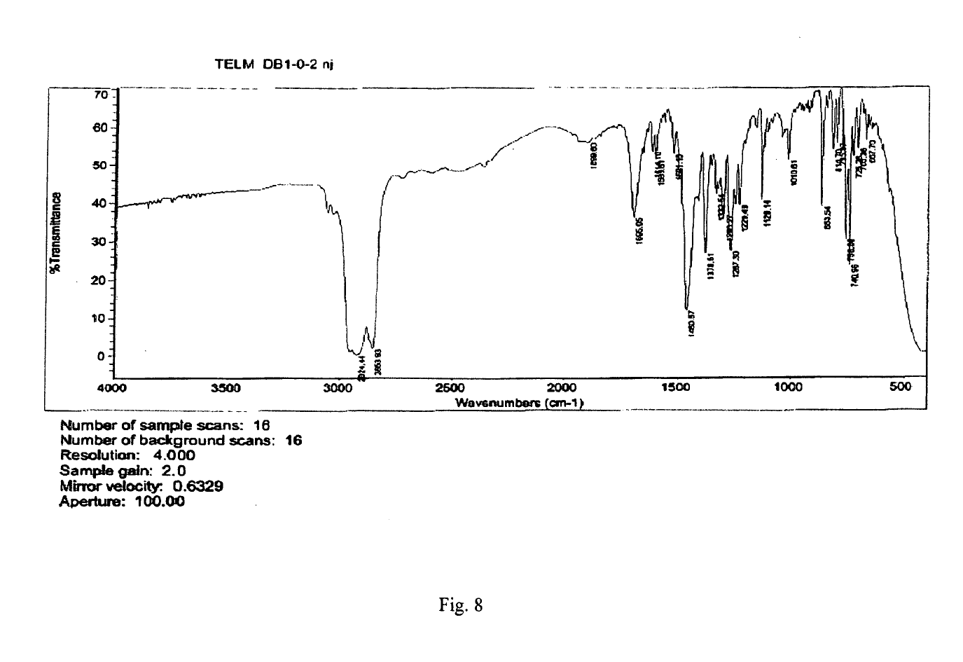

According to another embodiment of the present invention, the crystalline Telmisartan form A, obtained by crystallization from DMF, is further characterized by having an infra-red spectrum as shown in FIG. 8.

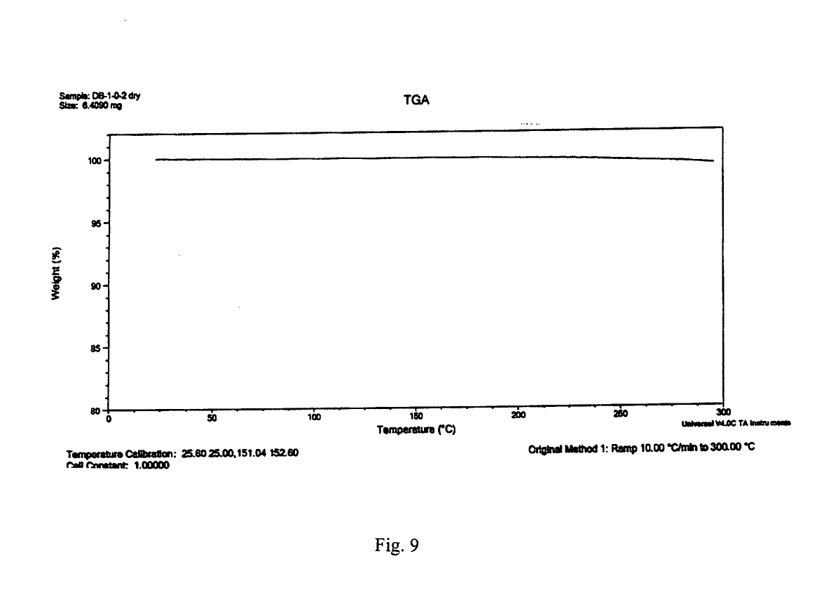

According to another embodiment of the present invention, the crystalline Telmisartan form A, obtained by crystallization from DMF, is further characterized by having a TGA curve as shown in FIG. 9.

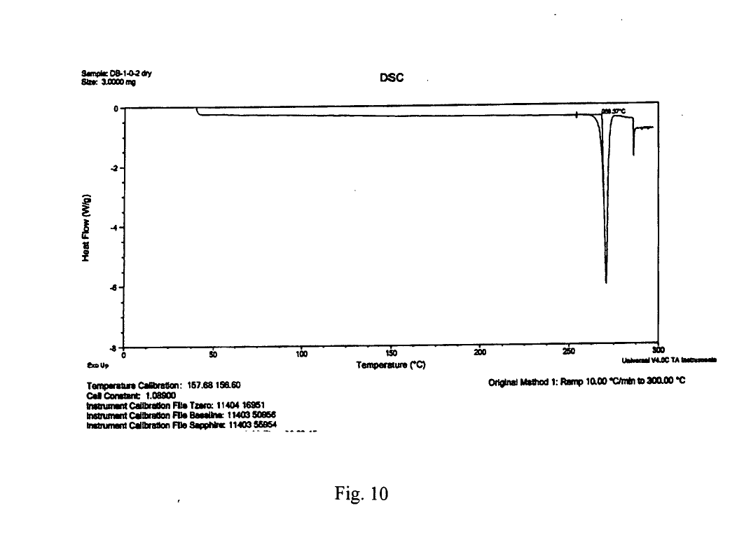

According to another embodiment of the present invention, the crystalline Telmisartan form A, obtained by crystallization from DMF, is further characterized by having a DSC curve as shown in FIG. 10.

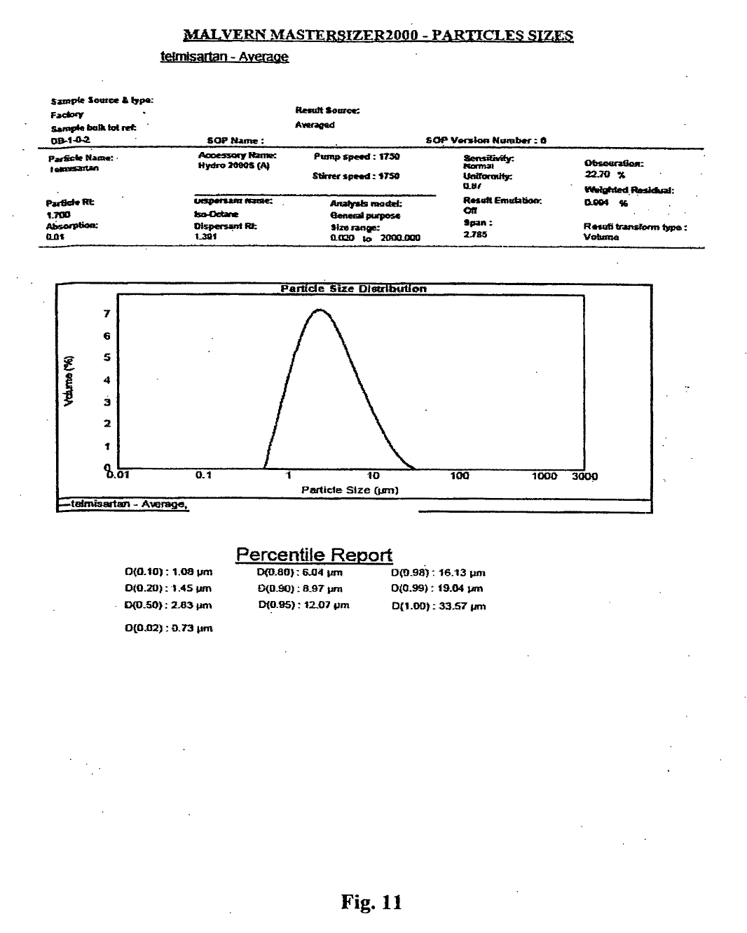

According to another embodiment of the present invention, the crystalline Telmisartan form A, obtained by crystallization from DMF, is further characterized by an average relatively small particle size distribution of less than 3μ for about 50% of the particles, as depicted in FIG. 11, hence the material shows excellent flowability.

According to a preferred embodiment of the present invention, there are provided novel processes for preparing Telmisartan form A, which is suitable for pharmaceutical use, by crystallization from a polar organic solvent e.g., N,N-dimethylformamide (DMF), N,N-dimethylacetamide (DMA), or N-methyl-2-pyrrolidone (NMP).

According to one aspect of the present invention, the Telmisartan starting material used in the process for preparing Telmisartan form A, may be either in dry state or in wet state.

According to the present invention, the term “dry” means that the material is substantially free of water, while the term “wet” means that the material contains substantial amount of water.

According to a preferred embodiment of the above-mentioned aspect of the present invention, the process for producing Telmisartan form A by crystallization from a polar organic solvent e.g., N,N-dimethylformamide (DMF), N,N-dimethylacetamide (DMA), or N-methyl-2-pyrrolidone (NMP), using dry Telmisartan, comprises:

providing a mixture of dry Telmisartan, obtained as per any suitable method including the method described in reference example 1, in the polar organic solvent while heating to elevated temperature;

cooling the mixture for sufficient time period to allow crystallization; and

filtering off the crystals, washing and drying, optionally at elevated temperature.

According to another embodiment of the present invention, the solution of Telmisartan in a polar organic solvent is heated to elevated temperature, preferably to about 65° C. and more preferably to about 90° C. or higher.

According to another embodiment of the present invention, the solvent used for washing the obtained crystals is selected from the group of C₁-C₄ alcohols, water and mixtures thereof, preferably ethanol.

According to another embodiment of the present invention, the obtained crystals are dried at elevated temperature, preferably at 100° C., and optionally under vacuum.

According to another embodiment of the present invention, the Telmisartan form A, prepared by the process described herein, is solvent free namely it contains residual solvents at a level of less than 5000 ppm, preferably of less than 1000 ppm and has LOD value of less than 0.5%, preferably of less than 0.3%, as measured by means of TGA.

According to another embodiment of the present invention, the Telmisartan form A, obtained by the process described herein, has a purity equal to or greater than 99.5%, and preferably it has a purity equal to or greater than 99.8%, which makes it suitable for pharmaceutical compositions.

In another preferred embodiment of the above-mentioned aspect of the present invention, there is provided a process for preparing Telmisartan form A by crystallization from a polar organic solvent e.g., N,N-dimethylformamide (DMF), N,N-dimethylacetamide (DMA), or N-methyl-2-pyrrolidone (NMP), using a wet Telmisartan, the process comprising:

dispersing a wet Telmisartan, obtained as per any suitable method including the method described in reference example 1, in a solvent mixture containing toluene and the polar organic solvent;

heating the dispersion to elevated temperature and collecting the water at this temperature by a Dean Stark fitting;

distilling off the toluene and filtering the hot mixture;

cooling the mixture sufficiently and filtering off the crystals; and

washing with an organic solvent and drying the crystals, optionally at elevated temperature.

According to another embodiment of the present invention, the wet Telmisartan is dispersed in a solvent mixture containing toluene and a polar organic solvent e.g., N,N-dimethylformamide (DMF), N,N-dimethylacetamide (DMA), or N-methyl-2-pyrrolidone (NMP) and heated to a temperature in the range from about 50° C. to reflux, preferably to an internal temperature of about 143° C.

According to another embodiment of the present invention, the solvent used for washing the obtained crystals is selected from the group of C₁-C₄ alcohols, water and mixtures thereof, preferably ethanol.

According to another embodiment of the present invention, the obtained crystals are dried at elevated temperature, preferably at 80° C., and optionally under vacuum.

According to another embodiment of the present invention, the Telmisartan form A, prepared by the process described herein is solvent free, namely it contains residual solvents at a level of less than 5000 ppm, preferably of less than 1000 ppm and has LOD value of less than 0.5%, preferably of less than 0.3%, as measured by means of TGA.

According to another embodiment of the present invention, the Telmisartan form A, obtained by the process described herein, has a purity equal to or greater than 99.5%, which makes it suitable for pharmaceutical formulations.

According to another preferred embodiment of the present invention, upon precipitation of Telmisartan from an aqueous solution containing an acid e.g., acetic acid, highly pure Telmisartan form A is provided in high yield, which equal to or greater than 93%.

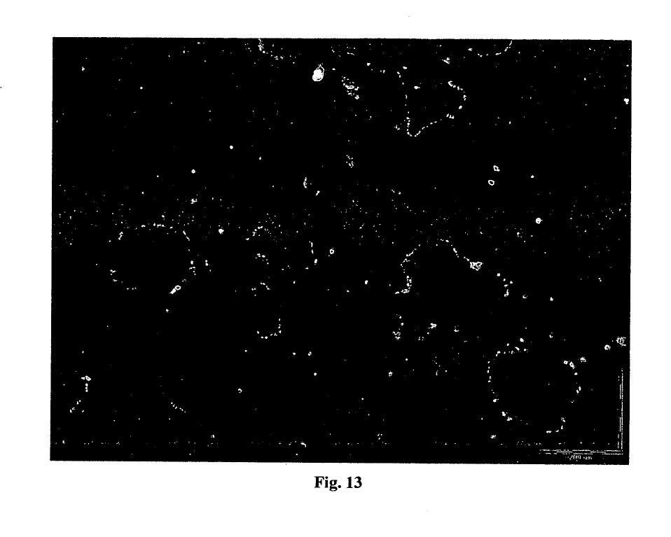

According to another preferred embodiment of the present invention, the Telmisartan form A, prepared by precipitation from aqueous solutions containing an acid, has a different crystal shape than needles namely the form of bulky shape as depicted in FIG. 13, hence the product may be readily filtered, it does not have a tendency to gain electrostatic charge upon grinding, and its flowability makes it suitable for industrial processing and scaling-up.

According to the teachings the present invention, Telmisartan form A is crystallized from a solvent selected from the group consisting of dimethyl sulfoxide (DMSO), N,N-dimethylformamide (DMF), N,N-dimethylacetamide (DMA), N-methyl-2-pyrrolidone (NMP), water, and mixtures thereof.

It is well known to those skilled in the art that while using a high boiling point solvent for crystallization e.g., dimethyl sulfoxide (DMSO), N,N-dimethylformamide (DMF), N,N-dimethylacetamide (DMA), or N-methyl-2-pyrrolidone (NMP), residual solvent is liable to remain in the final product after crystallization.

The inventors of the present invention have surprisingly discovered that while precipitating Telmisartan (obtained by crystallization from a high boiling point solvent), from an aqueous solution containing an acid, a highly pure solvent-free Telmisartan A is obtained.

According to yet another embodiment of the present invention, the Telmisartan form A, prepared by the process described herein, is solvent free namely it contains residual solvents at a level of less than 5000 ppm, preferably of less than 1000 ppm and has LOD value of less than 0.5%, preferably of less than 0.3%, as measured by means of TGA.

According to a preferred embodiment of the present invention, the process for producing Telmisartan form A by precipitation from aqueous solutions, comprises:

providing a mixture of Telmisartan, obtained as per any suitable method including the method described in reference example 1, and water while heating;

adding a base and optionally filtering the insoluble matter;

adding an acid to the filtrate to form a suspension and stirring; and

isolating the precipitated crystals.

According to another embodiment of the present invention, the acid is an inorganic acid or an organic acid selected from the group consisting of acetic acid, propionic acid, citric acid, maleic acid, fumaric acid and combinations thereof, preferably acetic acid.

According to another embodiment of the present invention, the mixture of Telmisartan with water is heated to elevated temperature, preferably to about 65° C. and more preferably to about 85° C.

According to another embodiment of the present invention, the solvent used for washing the obtained crystals is selected from the group of C₁-C₄ alcohols, water and mixtures thereof, preferably water.

According to another embodiment of the present invention, the obtained crystals are dried at elevated temperature, preferably at 80° C., and optionally under vacuum.

According to another embodiment of the present invention, the base is selected from the group consisting of ammonia, sodium hydroxide, potassium hydroxide, sodium carbonate, potassium carbonate and combinations thereof, preferably ammonia, e.g., as a 28% solution in water.

According to another embodiment of the present invention, the Telmisartan form A, obtained by the process described herein, has a purity equal to or greater than 99.5%, and preferably a purity equal to or greater than 99.8%, which makes it suitable for pharmaceutical compositions.

### EXAMPLES

Experimental Methods:

Optical Microscopy:

Microscope pictures were taken using Olympus BX50, equipped with UPlan FI objectives, and C-3030 Zoom digital camera (Olympus). Software: DPSoft version 3.1.

Average Particle Size Measurement:

Particle size was measured by Malvern model Mastersizer 2000, equipped with Malvern Hydro G circulation cell. Carrier: liquid:water.

System description: Measuring range: from 0.02-2000 μm. Accuracy: 1% on the Dv50 Light sources: Red light-helium neon laser. Blue light-solid state light source.

X-Ray Powder Diffraction Data (XRPD):

X-ray powder diffraction data (XRPD) were acquired using a PHILIPS X-ray diffractometer model PW1050-70. System description: Kα1=1.54178 Å, V=40 kV, 1=28 mA, diversion slit=1°, receiving slit=0.2 mm, scattering slit=1° with a Graphite monochromator.

Thermogravimetric Analysis (TGA):

Thermogravimetric analysis was performed by using TA Instruments thermogravimetric analyzer model Q500, equipped with TGA autosampler and mass flow controllers.

Differential Scanning Calorimetry (DSC):

Differential scanning calorimetry (DSC) was run on TA instruments model Q1000, with Universal software version 3.88. Samples were analyzed inside crimped 40 μl Aluminum pans. Heating rate for all samples was 5° C./min.

Bulk Density:

Bulk density was measured using tapped density tester dual-platform model VanKel 50-2300 (Varian Inc.).

System specifications. Speed: nominal rate of 300 taps per minute, Accuracy: Actual setting±1 tap, Drop height: 14±2 mm. Platform rotation: 5-15 rotations/minute.

HPLC Analysis:

Column: Inertsil ODS-3, 5μ, 250×4.6 mm (GL Sciences Cat. No.: 5020-01732). Mobile phase: 70% methanol and 30% water containing 10 ml triethylamine per 1.0 liter, pH adjusted to 3.0 using phosphoric acid.

Flow rate: 1 ml/min; UV detection: 226 nm; Oven temperature: 35° C.

### Reference Example 1

In a 1000 ml three-necked round bottom flask equipped with a reflux condenser, a thermometer and a magnetic stirrer, 4-[(1,4′-dimethyl-2′-propyl[2,6′-bi-1H-benzimidazol]-1′-yl)methyl]-[1,1′-biphenyl]-2-carboxylic acid methyl ester (50 g, 0.095 mole) was charged, and methanol (300 ml) was added followed by addition of water (26 ml) and 47% NaOH solution (27 ml). The mixture was refluxed for 2 hours. Part of the solvent was evaporated and water (500 ml) was added in portions at 85° C. to afford a solution. The insoluble matter was removed by filtration and the mixture was neutralized with a solution of acetic acid (31.7 ml) in water (75 ml). The thus obtained crude Telmisartan cake was collected by filtration and washed with water to obtain 150 g of wet Telmisartan, which was dried under vacuum to obtain 48 g of the crude product in 98.6% yield, having a purity of 97%.

### Example 1

In a 500 ml three necked round bottom flask equipped with a reflux condenser, a thermometer and a magnetic stirrer, Telmisartan (15 g) was dissolved in DMSO (290 ml). The solution was heated to 65° C. using an oil bath, and left to cool down to 25° C. After few days the resulting crystals were filtered off, washed with fresh water and dried at 100° C. under vacuum to obtain 11.55 g of Telmisartan form A in 77% yield, having a purity of 99.5% (according to HPLC).

Depending on the drying strength, different batches of dried material contained residual DMSO level in the range of 500-1000 ppm and had LOD values in the range of 0.2-0.3%, as measured by means of TGA.

The obtained solid material contained lumps, which could be easily ground by means of conventional mill. The obtained ground material has improved flowability, namely it is a free flowing crystalline powder that does not tend to gain electrostatic charge upon grinding. It has a bulk density of about 0.3 g/ml.

Particle size distribution was found to be: D(v,0.1)=1.60μ; D(v,0.2)=2.25μ; D(v,0.5)=4.98μ; D(v,0.8)=11.46μ; D(v,0.9)=16.78μ; D(v,0.95)=22.21μ; D(v,0.98)=29.03μ; D(v,1.0)=52.87μ.

XRPD pattern of the resulting material is shown in FIG. 4 and it resembles the pattern of form A.

### Example 2

In a 1 liter three-necked round bottom flask equipped with a reflux condenser, a thermometer and an upper stirring rod, Telmisartan (15 g) and magnesium stearate (0.15 g) were dispersed in water (500 ml). The dispersion was heated to 80-90° C. and left with stirring for four days. Then, the mixture was filtered off by suction and dried under vacuum at 50° C. (Yield: 84%)

Particle size distribution was found to be: D(v,0.1)=0.35μ; D(v,0.2)=0.85μ; D(v,0.5)=4.33μ; D(v,0.8)=11.90μ; D(v,0.9)=17.48μ; D(v,0.95)=23.03μ; D(v,0.98)=30.08μ; D(v,1.0)=52.871μ.

XRPD pattern of the resulting material is depicted in FIG. 5 and resembles the pattern of form A.

### Example 3

A reaction vessel equipped with a Dean Stark fitting (filled with toluene) was charged with wet Telmisartan (150 g, which are 48 g on dry basis), DMF (293 ml) and toluene (293 ml). The mixture was stirred and heated in an oil bath at 150° C. and water (about 108 ml) was collected in the Dean Stark fitting. The Dean Stark fitting was removed and the solvent was distilled off (about 300 ml of toluene distillate was collected, while the internal temperature rose to about 143° C.). The hot mixture was filtered and the hot filtrate was transferred to a clean vessel. The mixture was cooled to 25° C. while stirring, during which time crystallization occurred. Stirring was maintained at 25° C. for 1 hour and then the mixture was cooled to 5° C. Stirring was maintained at 5° C. for 1 hour. The crystals were obtained by filtration and washed with cold ethanol, and dried at 80° C. in vacuum to afford 36 g of dry crystallized Telmisartan in 75% yield, having a purity of 99.5%.

### Example 4

In a 500 ml three-necked round bottom flask equipped with a reflux condenser, a thermometer and a magnetic stirrer, Telmisartan (58.4 g) was suspended in DMF (293 ml). The suspension was heated to 90° C. using an oil bath, and left to cool down to 25° C. Mixing was maintained at this temperature for about an hour. Then, the mixture was cooled down to 5° C. and mixing was maintained at this temperature for about an hour. The solid was obtained by filtration, washed with cold ethanol and dried under vacuum to afford 47.9 g of the dried material in 82% yield, having a purity of 99.9%.

Particle size distribution was found to be: D(v,0.1)=1.08; D(v,0.2)=1.45; D(v,0.5)=2.83μ; D(v,0.8)=6.04μ; D(v,0.9)=8.97μ; D(v,0.95)=12.07μ; D(v,0.98)=16.13μ; D(v,1.0)=33.57μ.

### Example 5

In a 500 ml three-necked round bottom flask equipped with a reflux condenser, a thermometer and a magnetic stirrer, Telmisartan (15 g) was suspended in DMA (290 ml). The suspension was heated to 90° C. using an oil bath, and left to cool down to 25° C. Mixing was maintained at this temperature for about an hour. Then, the mixture was cooled down to 5° C. and mixing was maintained at this temperature for about an hour. The solid was obtained by filtration, washed with cold ethanol and dried under vacuum to afford the dried material having a purity of 99.9%.

### Example 6

In a 500 ml three-necked round bottom flask equipped with a reflux condenser, a thermometer and a magnetic stirrer, Telmisartan (15 g) was suspended in NMP (290 ml). The suspension was heated to 90° C. using an oil bath, and left to cool down to 25° C. Mixing was maintained at this temperature for about an hour. Then, the mixture was cooled down to 5° C. and mixing was maintained at this temperature for about an hour. The solid was obtained by filtration, washed with cold ethanol and dried under vacuum to afford the dried material having a purity of 99.7%.

### Example 7

In a 250 ml three-necked round bottom flask equipped with a reflux condenser, a thermometer and a magnetic stirrer, Telmisartan (9.8 g) was mixed with water (98 ml) under stirring and heated to 85° C. to afford a suspension. 28% ammonia solution (22.5 ml) was added in portions at 85° C. On completion of the addition a clear solution was obtained. Filtration was carried out and the hot filtrate was transferred to a clear reaction vessel. A solution of acetic acid (18.2 ml) in water (43 ml) was added in portions at 85° C. to afford a suspension and the pH was checked (an optimal pH value should be in the range of 4-5). The suspension was stirred at 85° C. for 15 minutes and filtered. The cake was washed with hot water and dried at 80° C. in vacuum to afford 9.1 g of dry precipitated Telmisartan in 93% yield, having a purity of 99.5%.

The results of particle size distribution before and after milling are presented in Table 1.

TABLE 1

| Description | D(V, 0.1) | D(V, 0.5) | D(V, 0.9) |
|---|---|---|---|
| Un-milled material | 51.4 | 245.0 | 578.2 |
| Ground material | 0.7 | 3.3 | 19.1 |
| Sonicated material with | 0.8 | 8.1 | 24.7 |
| no milling | | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A crystalline solid comprising Telmisartan form A suitable for pharmaceutical formulations, characterized by having improved flowability, ease of filtration from its crystallization medium, and having low tendency to gain electrostatic charge upon grinding.

2. A process for preparing the crystalline solid comprising Telmisartan form A of claim 1, by crystallization of Telmisartan from a polar organic solvent selected from the group consisting of dimethyl sulfoxide (DMSO), N,N-dimethylformamide (DMF), N,N-dimethylacetamide (DMA), N-methyl-2-pyrrolidone (NMT), water, and mixtures thereof.

3. The crystalline solid comprising Telmisartan form A of claim 1, prepared by crystallization from DMSO, characterized by having a substantial amount of prismatic crystals with the longest dimension shorter than about 100 μm, as measured by means of optical microscopy.

4. The crystalline solid comprising Telmisartan form A of claim 3, further characterized by having bulk density of about 0.3 g/ml.

5. The process of claim 2 for preparing the crystalline solid comprising Telmisartan form A by crystallization from DMSO, comprising:
dissolving Telmisartan, obtained as per any suitable method including the method described in reference example 1, in DMSO while heating the mixture to elevated temperature;
cooling the solution for sufficient time to allow crystallization; and
filtering off the crystals, washing and drying.

6. The process of claim 5, wherein the crystalline solid comprising Telmisartan form A contains residual DMSO at a level of less than 1000 ppm.

7. The process of claim 5, wherein the crystalline solid comprising Telmisartan form A has LOD value of less than 0.3%, as measured by means of TGA.

8. The process of claim 2 for preparing crystalline solid comprising Telmisartan form A, by crystallization from water, comprising:
dispersing Telmisartan, obtained as per any suitable method including the method described in reference example 1, in hot water, optionally with addition of magnesium stearate;
stirring for an extended time, e.g., for about four days at elevated temperature;
cooling gradually (e.g., slowly) to ambient temperature; and
isolating the crystals by filtration and drying.

9. The process of claim 8, wherein the crystalline solid comprising Telmisartan form A, characterized by having bulk density of about 0.22 g/ml.

10. The process of claim 8, wherein the temperature of the hot water is about 80-90° C.

11. The process of claim 8, wherein the amount of magnesium stearate is about 1-2%.

12. The process of claim 2 for preparing the crystalline solid comprising Telmisartan form A by crystallization from a polar solvent using dry Telmisartan, comprising:
providing a mixture of dry Telmisartan, obtained as per any suitable method including the method described in reference example 1, in a polar organic solvent while heating to elevated temperature;
cooling the mixture for a sufficient time period to allow crystallization; and
filtering off the crystals, washing and drying, optionally at elevated temperature.

13. The process of claim 12, wherein the polar organic solvent is selected from the group consisting of N,N-dimethylformamide (DMF), N,N-dimethylacetamide (DMA), and N-methyl-2-pyrrolidone (NMP).

14. The process of claim 12 for preparing the crystalline solid comprising Telmisartan form A, wherein the solution of Telmisartan in the polar organic solvent is heated to elevated temperature of about 90° C.

15. The process of claim 12 for preparing the crystalline solid comprising Telmisartan form A, wherein the solvent used for washing the obtained crystals is selected from the group of C₁-C₄ alcohols, water, and mixtures thereof.

16. The process of claim 15 for preparing the crystalline solid comprising Telmisartan form A, wherein the solvent used for washing the obtained crystals is ethanol.

17. The process for preparing the crystalline solid comprising Telmisartan form A of claim 2 by crystallization form a polar organic solvent, e.g., N,N-dimethylformamide (DMF), N,N-dimethylacetamide (DMA), or N-methyl-2-pyrrolidone (NMP) using wet Telmisartan, comprising:
dispersing the wet Telmisartan, obtained as per any suitable method including the method described in reference example 1, in a solvent mixture containing toluene and the polar organic solvent;
heating the dispersion to elevated temperature and collecting the water at this temperature by a Dean Stark fitting;
distilling off the toluene and filtering the hot mixture;
cooling the mixture sufficiently and filtering off the crystals; and
washing with an organic solvent and drying the crystals, optionally at elevated temperature.

18. The process of claim 17 for preparing the crystalline solid comprising Telmisartan form A, wherein the wet Telmisartan is dispersed in a solvent mixture containing toluene and the polar organic solvent and heated to an internal temperature of about 143° C.

19. The process of claim 17 for preparing the Telmisartan form A, wherein the solvent used for washing the obtained crystals is selected from the group of C₁-C₄ alcohols, water, and mixtures thereof.

20. The process of claim 19 for preparing the Telmisartan form A, wherein the solvent used for washing the obtained crystals is ethanol.

21. The crystalline solid comprising Telmisartan form A of claim 1, prepared by precipitation from an aqueous solution, characterized by having a crystal shape which is not in the form of needles, but in the form of bulky shape, which makes it suitable for pharmaceutical compositions.

22. A process for preparing the crystalline solid comprising Telmisartan form A of claim 21 by precipitation from an aqueous solution, comprising:
providing a mixture of Telmisartan, obtained as per any suitable method including the method described in reference example 1, and water while heating;
adding a base and optionally filtering the insoluble matter;
adding an acid to the filtrate to form a suspension and stirring; and
isolating the precipitated crystals.

23. The process of claim 22 for preparing the crystalline solid comprising Telmisartan form A, wherein the aqueous solution of Telmisartan is heated to elevated temperature of about 85° C.

24. The process of claim 22 for preparing the crystalline solid comprising Telmisartan form A, wherein the solvent used for washing the obtained crystals is selected from the group consisting of C₁-C₄ alcohols, water, and mixtures thereof.

25. The process of claim 24 for preparing the crystalline solid comprising Telmisartan form A, wherein the solvent used for washing the obtained crystals is water.

26. The process of claim 22 for preparing the crystalline solid comprising Telmisartan form A, wherein the base is selected from the group consisting of ammonia, sodium hydroxide, potassium hydroxide, sodium carbonate, potassium carbonate, and combinations thereof.

27. The process of claim 26 for preparing the crystalline solid comprising Telmisartan form A, wherein the base is 28% ammonia solution.

28. The process of claim 22 for preparing the crystalline solid comprising Telmisartan form A, wherein the acid is an inorganic acid or an organic acid selected from the group consisting of acetic acid, propionic acid, citric acid, maleic acid, fumaric acid and combinations thereof.

29. The process of claim 28 for preparing the crystalline solid comprising Telmisartan form A, wherein the acid is acetic acid.

30. Telmisartan form A having a purity equal to or greater than 98%.

31. Telmisartan form A having a purity equal to or greater than 99.5%.

32. Telmisartan form A in which 90% of the particles have a diameter of 60 microns or less, i.e., d(0.9) is equal to or less than 60 microns.

33. Telmisartan form A in which 90% of the particles have a diameter of 18 microns or less, i.e., d(0.9) is equal to or less than 18 microns.

34. A pharmaceutical composition comprising the crystalline solid comprising Telmisartan form A of claim 1 and pharmaceutically acceptable excipients and additives.
